(19) United States
(12) Patent Application Publication
(10) Pub. No.: US 2018/0110735 A1
(43) Pub. Date: Apr. 26, 2018

(54) ANTIMICROBIAL ALGINATE-BASED MICROPARTICLES AND RELATED MATERIALS AND METHODS

(71) Applicant: Bard Access Systems, Inc., Salt Lake City, UT (US)

(72) Inventors: James Freasier, Salt Lake City, UT (US); Lindsey Corum, Salt Lake City, UT (US); Emir Rahislic, Salt Lake City, UT (US)

(21) Appl. No.: 15/727,881

(22) Filed: Oct. 9, 2017

Related U.S. Application Data
(60) Provisional application No. 62/410,652, filed on Oct. 20, 2016.

(51) Int. Cl. A61K 9/16; A61K 31/155; A61K 9/12; A61L 15/44; A61L 15/28; A61L 15/20

(52) U.S. Cl. CPC A61K 9/1652; A61K 31/155; A61K 9/1623; A61K 9/122; A61L 2300/404; A61L 15/44; A61L 15/28; A61L 15/20; A61K 9/1682

(57) ABSTRACT

Microparticles that include a charged polysaccharide (e.g., alginate) and an antimicrobial agent (e.g., chlorhexidine), along with related compositions and methods. The microparticles and related compositions can provide antimicrobial properties. Methods for manufacturing such microparticles, along with methods for applying the resulting microparticles to a substrate, such as a medical device or dressing, are also disclosed.

### RELATED APPLICATIONS

This application claims priority to U.S. Provisional Application No. 62/410,652, filed Oct. 20, 2016, and titled ANTIMICROBIAL ALGINATE-BASED MICROPARTICLES AND RELATED MATERIALS AND METHODS, the entirety of which is incorporated herein by reference.

### TECHNICAL FIELD

The present disclosure relates generally to compositions having antimicrobial properties, along with related methods. More particularly, some embodiments of the disclosure relate to alginate-based microparticles, such as alginate-based microparticles with antimicrobial properties. Related materials and methods are also disclosed.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1A is a perspective view of a dressing that includes a plurality of microparticles prior to being applied to a wound bed.

FIG. 1B is a perspective view of the dressing of FIG. 1A after the dressing has been applied to a wound bed.

FIG. 2 is a graph showing the elution rate of chlorhexidine from various microparticles.

FIG. 3 is a scatterplot showing the relationship between melting point and chlorhexidine elution rate for various sets of microparticles.

FIG. 4 is an SEM image showing microparticles at 500× magnification.

FIG. 5 is an SEM image of the microparticles of FIG. 4 at 3000× magnification.

FIG. 6 is a graph providing size distribution curves for the microparticles depicted in FIGS. 4 and 5.

### DETAILED DESCRIPTION

The present disclosure relates generally to compositions having antimicrobial properties, along with related methods. More particularly, some embodiments of the disclosure relate to alginate-based microparticles, such as alginate-based microparticles with antimicrobial properties. Related materials and methods are also disclosed.

Compositions with antimicrobial and/or hemostatic properties can be useful in certain medical contexts. For example, in many medical procedures, a medical instrument is percutaneously inserted into a patient. To prevent external bleeding and/or infection, the resulting wound may be treated with one or more compositions that provide antimicrobial protection and/or facilitate blood coagulation. For example, a dressing that includes one or more hemostatic and/or antimicrobial agents may be applied at or adjacent to the site of the wound, thereby reducing surface bleeding and providing protection against infection. More particularly, in some embodiments, the dressing for the wound may include an absorptive material that is impregnated and/or coated with microparticles that improve the antimicrobial and/or hemostatic properties of the dressing.

In other circumstances, a composition with antimicrobial properties may be applied to portions of a medical device, such as a portion of catheter that is typically disposed adjacent to or proximal of a wound bed when the catheter is in use. For example, an antimicrobial and/or hemostatic composition may be coated onto the cuff of a catheter that is designed to permit tissue granulation into the cuff to anchor the cuffed catheter to the patient. The hemostatic and/or antimicrobial composition may prevent or reduce surface bleeding and decrease the likelihood of infection resulting from use of the cuffed catheter. The aforementioned uses are merely exemplary uses for compositions with antimicrobial and/or hemostatic properties, and are not intended to limit the scope of this disclosure. Indeed, other uses for the compositions described herein are also contemplated.

More generally, it will be readily understood that the embodiments, as generally described herein, are exemplary. Thus, the following more detailed description of various embodiments is not intended to limit the scope of the present disclosure, but is merely representative of various embodiments.

The term “microparticle” refers to any particle having a diameter of 100 nm to 1000 μm. A “substantially spherical” microparticle (i.e., a microsphere) is a microparticle having a sphericity of greater than 0.9. Unless otherwise specified, the “diameter” of an irregularly shaped microparticle is the average diameter of the microparticle (i.e., the diameter of a sphere of equivalent volume). The term “half-life” refers to the period of time in which a quantity decreases by half, even if the decrease is not exponential. The melting point of the “alginate of the microparticles” refers to the temperature at which the alginate melts when incorporated into the microparticles. The hydrophobic-lipophilic balance of a surfactant is determined using Griffin's method. Unless otherwise specified, all ranges include both endpoints.

One aspect of this disclosure relates to compositions that include a plurality of microparticles. In some embodiments, the microparticles may include a hydrophilic polysaccharide, such as chitosan, alginate, heparin, hyaluronic acid, or pectin. The hydrophilic polysaccharide may be positively or negatively charged. In some instances, the polysaccharide is negatively charged. For example, in some embodiments, the negatively charged polysaccharide is an alginate, a block copolymer that includes blocks of (1-4)-linked β-D-mannuronate and α-L-guluronate residues. In some embodiments, the alginate has a weight average molecular weight of between 50,000 Da and 350,000 Da and/or between 100,000 Da and 200,000 Da. Without being bound to any particular theory, alginate (or another hydrophilic polysaccharide of the microparticles) may initiate or further a clotting cascade that promotes coagulation of blood. Stated differently, the polysaccharide (e.g., alginate) may provide a structural scaffold to facilitate clot formation and control surface bleeding. In some embodiments, the microparticles are, on average, between about 15% and about 30% polysaccharide (e.g., alginate) by weight. In some embodiments, the microparticles are, on average, between about 20% and about 25% polysaccharide (e.g., alginate) by weight.

In some embodiments, the microparticles may further include an antimicrobial agent. More specifically, in some embodiments, the antimicrobial agent is a positively charged antimicrobial agent, such as chlorhexidine. For example, in some embodiments, the microparticles include a chlorhexidine salt, such as chlorhexidine acetate, chlorhexidine hydrochloride, or chlorhexidine gluconate. In some embodiments, the microparticles are, on average, between about 25% and about 45% antimicrobial agent (e.g., chlorhexidine) by weight. In some embodiments, the microparticles are, on average, between about 30% and about 35% antimicrobial agent (e.g., chlorhexidine) by weight. In some embodiments, the antimicrobial agent is effective against both gram-positive and gram-negative bacteria. In some embodiments, the antimicrobial agent is a fungicide. In certain embodiments, the antimicrobial agent provides fungicidal activity in addition to its antimicrobial properties.

In some embodiments, the microparticles further include one or more surfactants. For example, in some embodiments, the microparticles include a surfactant that has a hydrophobic-lipophilic balance of between 9 and 17 or between 11 and 15. In some embodiments, the surfactant is a polysorbate, such as polysorbate 80. In some embodiments, other surfactants may be used, such as polysorbate 20, polysorbate 40, polysorbate 60, polysorbate 65, polysorbate 85, polyoxyethylene monostearate, and/or polyethylene glycol 400 monostearate. In some embodiments, the microparticles are, on average, between about 25% and about 45% surfactant by weight. For example, in some embodiments, the microparticles are, on average, between about 30% and 45% surfactant by weight, between about 35% and 45% surfactant by weight, and/or between about 40% and 45% surfactant by weight.

In some embodiments, the microparticles may have an alginate shell or layer that encapsulates at least a portion of the antimicrobial agent. For example, in some embodiments, the microparticles have a sodium alginate shell that encapsulates chlorhexidine (e.g., chlorhexidine gluconate). In some embodiments, the antimicrobial agent may be dispersed (e.g., uniformly dispersed or substantially uniformly dispersed) throughout an alginate matrix that forms the microparticles. And in certain of such embodiments, an alginate shell or layer may be formed on or around the alginate matrix, forming an exterior layer on the microparticles.

In some embodiments, the microparticles are of relatively uniform size, while in other embodiments, the microparticles differ significantly in size. The median diameter of the microparticles may be between 100 nm and 1000 μm, between 200 nm and 900 μm, between 500 nm and 700 μm, between 750 nm and 500 μm, between 1 μm and 250 μm, between 1 μm and 100 μm, between 100 nm and 1 μm, between 100 nm and 20 μm, between 100 nm and 500 μm, between 500 μm and 1000 μm, or between 250 μm and 1000 μm.

In some embodiments, the polysaccharide of the microparticles (e.g., the alginate) has a melting point of between 130° C. and 180° C. Stated differently, alginate that is incorporated into the microparticles may melt when heated to between 130° C. and 180° C. In some embodiments, the alginate of the microparticles has a melting point of greater than or equal to 160° C. In other embodiments, the alginate of the microparticles has a melting point of less than 160° C.

In some embodiments, the microparticles are biocompatible. For example, the polysaccharide and/or other components of the microparticles can be biocompatible. Biocompatible materials are non-toxic to tissues and cells and generally do not cause inflammation. In some embodiments, the microparticles are biodegradable. For example, the polysaccharide and/or other components of the microparticles can be biodegradable. In further embodiments, degradation of the microparticles can result in the release of an antimicrobial agent incorporated therein. For example, chlorhexidine and/or another antimicrobial agent can be released as the microparticles degrade, or as the polysaccharide of the microparticles degrades.

In some embodiments, the microparticles are substantially spherical in shape. Stated differently, in some embodiments, the microparticles are microspheres. In other or further embodiments, the microparticles are shaped such that, when viewing any cross-section of the microparticle, the difference between the major diameter (or maximum diameter) and the minor diameter (or minimum diameter) is less than 20%, less than 15%, less than 10%, less than 5%, or less than 1%. In some embodiments, the microparticles may have a major diameter/minor diameter ratio of from about 1.0 to about 2.0, from about 1.0 to about 1.5, or from about 1.0 to about 1.2.

In some embodiments, the microparticles are substantially devoid of cellulose and oxidized cellulose, such as microdispersed oxidized cellulose. In some circumstances, microparticles that lack oxidized cellulose, such as micronized oxidized cellulose, may be cheaper and/or easier to manufacture than microparticles that include oxidized cellulose. However, in some embodiments, the microparticles may be cross-linked to oxidized cellulose, such as microdispersed oxidized cellulose.

In some embodiments, the microparticles are applied to or incorporated into a substrate, such as a dressing or a catheter (e.g., a catheter cuff). For example, in some embodiments, the microparticles are disposed on an outer surface of a substrate, such as a foam. In some embodiments, the microparticles are a component of a dressing for a wound. More particularly, in some embodiments, the microparticles are disposed on a surface of a foam (e.g., a polyurethane foam) and the resulting composition is applied to (or adjacent to) a wound bed. In some embodiments, the foam functions as sponge that is capable of absorbing multiples of its own weight (e.g., more than 5× and/or 10×) in fluid, exudate, and/or blood.

In some embodiments, all or substantially all of the microparticles of a composition are all derived from a single lot. Stated differently, all or substantially all of the microparticles of a composition may be manufactured using the same process.

In some embodiments, the amount of antimicrobial agent (e.g., chlorhexidine) in a dressing is sufficient to provide a four-log reduction against clinically relevant test organisms (e.g., methicillin-resistant staphylococcus aureus) as determined using the AATCC Test Method 100-2004. In some embodiments, the amount of antimicrobial agent (e.g., chlorhexidine) in a composition (e.g., a dressing) is less than 20 mg, less than 10 mg, less than 5 mg, and/or less than 1 mg, while still providing sufficient antimicrobial activity. For example, the microbial agent may provide sufficient antibacterial (e.g., a four-log reduction of activity) over a period of at least one week.

In some embodiments, the microparticles include one or more cross-linking agents that ionicially or covalently cross-link the alginate molecules. The cross-linking agents may cross-link alginate molecules in an intramolecular and/or intermolecular fashion. In some embodiments, the cross-linking agent is selected from the group consisting of Ca⁺², formaldehyde, and/or glutaraldehyde.

In some embodiments, the microparticles are designed to release antimicrobial agent (e.g., chlorhexidine) at a rate such that the half-life for antimicrobial release is between about 0.1 h and 60 h, between about 0.2 h and 60 h, between about 0.2 h and 40 h, and/or between about 0.5 h and 24 h when the microparticles are immersed in a solution of phosphate buffered saline (PBS). (Immersion in a PBS solution may result in release of an antimicrobial agent at a rate that is greater than the release rate of chlorhexidine from the device in typical clinical settings.)

Microparticles having antimicrobial and/or hemostatic properties may be manufactured in any suitable manner. For example, in some embodiments, the microparticles are formed via a spray drying process. In other embodiments, the microparticles are prepared by some other method (e.g., water-in-oil emulsions).

Some methods for manufacturing microparticles (or articles/compositions that include microparticles) may include the step of combining a polysaccharide (e.g., an anionic polysaccharide), an antimicrobial agent (e.g., a positively charged antimicrobial agent), a surfactant, and a liquid to form a mixture (e.g., a solution or slurry). The resulting mixture may then be spray dried to form a plurality of microparticles.

As noted above, the polysaccharide (e.g., anionic polysaccharide) may be an alginate, such as an alginate salt. For example, in some embodiments, sodium alginate may be combined with a surfactant, an antimicrobial agent, and a liquid to form a mixture that is used to generate microparticles. In some embodiments, the mixture, immediately prior to spray drying, is between 0.2% and 1.5% antimicrobial agent (w/v). In some embodiments, the weight average molecular weight of the alginate in the mixture is between 50,000 Da and 350,000 Da and/or between 100,000 Da and 200,000 Da.

In some embodiments, the antimicrobial agent (e.g., positively charged antimicrobial agent) is chlorhexidine. More particularly, in some embodiments, the chlorhexidine is a chlorhexidine salt, such as chlorhexidine acetate, chlorhexidine hydrochloride, or chlorhexidine gluconate. In some embodiments, the mixture is between about 0.2% and 2.5% (w/v). The surfactant may have a hydrophobic-lipophilic balance of between 9 and 17 or between 11 and 15. For example, in some embodiments, the surfactant is a polysorbate, such as polysorbate 80. In some embodiments, other surfactants may be used. In some embodiments, the mixture, immediately prior to spray drying, is between about 0.25% and about 1.0% surfactant (w/v). In some embodiments, the amount of surfactant in the mixture, immediately prior to spray drying of the mixture, is present at a concentration that exceeds the critical micelle concentration for the surfactant in the liquid.

In some embodiments, the liquid comprises and/or consists essentially of water. In some embodiments, the liquid comprises and/or consists essentially of an organic solvent. In some embodiments, the liquid includes both water and an organic solvent.

In some embodiments, a cross-linking agent may be added to the alginate salt prior to spray drying of the mixture. In some embodiments, the cross-linking agent is added to the alginate salt at the spray nozzle, or while spraying. The inclusion of the cross-linking agent may induce intramolecular and/or intermolecular cross-linking of the alginate. Such cross-linking may increase the stability of the microparticles and decrease the release rate of antimicrobial from the microparticles. In some embodiments, the cross-linking agent (e.g., divalent cations such as Ca⁺²) may produce an ionic cross-link, while in other embodiments, the cross-linking agent (e.g., glutaraldehyde and/or formaldehyde) produces a covalent cross-link.

In some instances, a mixture may be advanced through a spray nozzle or atomizer of a spray dryer to disperse the mixture into a plurality of droplets. Liquid may then be removed from the emerging droplets to yield spray-dried microparticles. For example, in some embodiments, spray drying the mixture removes more than 80%, more than 90%, and/or more than 95% of the liquid in the mixture.

The microparticles may be manufactured (e.g., spray dried) at any suitable temperature. For example, in some embodiments, a mixture of antimicrobial agent, alginate salt, surfactant, and liquid may be spray dried at an inlet temperature that is between about 105° C. and about 160° C. Spray drying feed rate may range from between about 1 mL/min and 10 mL/min.

In some embodiments, the resulting microparticles may have an alginate shell or exterior layer that encapsulates at least a portion of the antimicrobial agent (e.g., chlorhexidine). The shell may prevent or inhibit the release of the enclosed antimicrobial agent.

Microparticle size may be tailored by varying one or more parameters of the manufacturing process. For example, the size of microparticles formed via spray drying may be controlled by, inter alia, one or more of the following: (1) altering the concentration or relative amount of one or more components (e.g., the surfactant) in the mixture, (2) altering the delivery rate of the mixture into the spray dryer, and (3) altering the inlet temperature for the spray dryer.

In some embodiments, the microparticles manufactured as described above are then applied to or impregnated into a substrate. For example, in some embodiments, microparticles are applied to or impregnated into a dressing for a wound to improve the antimicrobial and/or hemostatic properties of dressing.

More particularly, in some embodiments, microparticles are applied only to a portion of the dressing that is configured for contact with a patient. For example, the microparticles may be applied only to a lower surface of the dressing. By applying the microparticles only to surfaces that are configured for contact with the patient, the dressing may provide sufficient antimicrobial efficacy at relatively low amounts of antimicrobial agent. Stated differently, as antimicrobial agent may become trapped within a substrate, such as a polyurethane foam dressing, when the antimicrobial agent is impregnated into the substrate, lower amounts of antimicrobial agent may be used when the antimicrobial agent is applied to, but not impregnated into, the substrate.

More particularly, in some embodiments, the microparticles may be applied to a substrate by spray coating, biocompatible adhesives, dip coating, and/or dry compounding in raw materials. In some instances, the microparticles may be applied to the substrate at relatively low temperatures, such as below 100° C. In some instances, chlorhexidine can potentially break down into toxic by-products, such as para-chloroaniline through thermal decomposition or hydrolysis at temperatures above 150° C. Manufacture of the microparticles and/or application or impregnation of the microparticles onto a substrate at low temperatures may provide various advantages. For example, at high temperatures, chlorhexidine may decompose into, among other breakdown products, 4-chloroaniline, a suspected genotoxin. Thus, manufacture of the microparticles and/or application or impregnation of the microparticles into a substrate at relatively low temperatures may decrease the amount of undesired 4-chloroaniline in the microparticles.

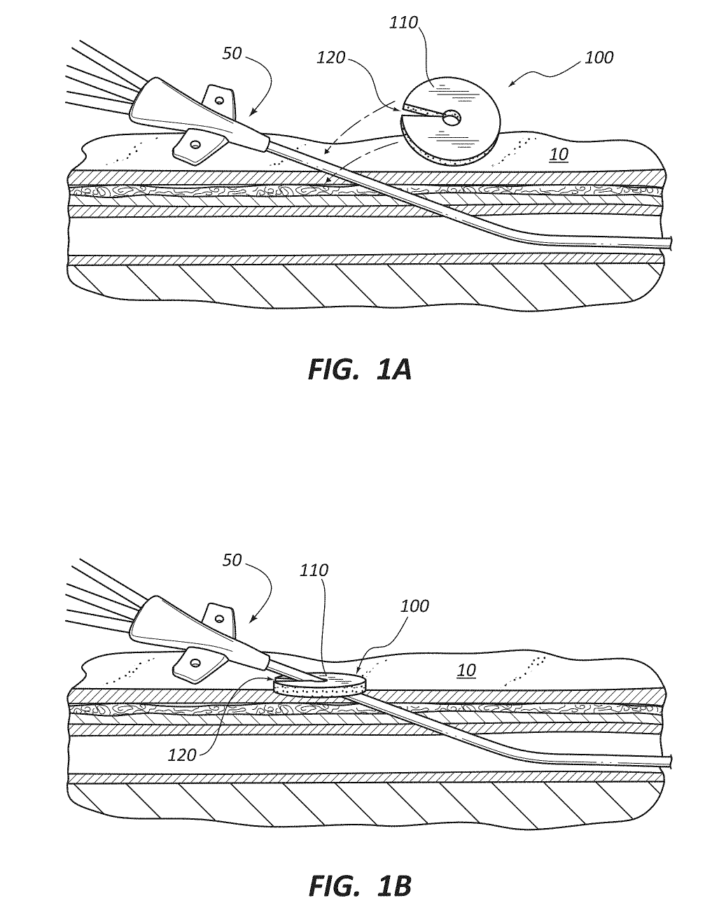

An exemplary dressing 100 that includes a plurality of microparticles is depicted in FIGS. 1A and 1B. As shown in these figures, the dressing 100 includes a generally disc-shaped foam 110 with a slit 120 that extends from the center of the disc to the outer edge of the disc. The slit 120 is designed to allow a practitioner to position the dressing 100 around an entry site for a percutaneously inserted medical device, such as a catheter 50. In other words, after insertion of the catheter 50 into a patient 10, the dressing 100 may be placed around a portion of the catheter 50 such that the lower surface of the dressing 100 is in contact with the skin and/or wound of the patient 10. A plurality of microparticles may be impregnated into and/or applied to a lower surface of the dressing 100. The microparticles may reduce surface bleeding and/or reduce the risk of infection at the wound site. Once the dressing 100 is applied as shown in FIG. 1B, an adhesive covering may be placed over the dressing 100 to secure the dressing 100 to the patient 10 and to further protect the wound site from the external environment. In some embodiments, the adhesive covering is transparent to allow visualization of the dressing and/or the percutaneously inserted medical device through the covering.

In other embodiments, the microparticles may be applied to or impregnated into one or more other substrates. For example, as noted above, microparticles may be used to coat a catheter cuff.

### EXAMPLES

### Example 1—Manufacture of Microparticles

A sodium alginate solution was made by adding 7 g of 100,000 g/mol sodium alginate to 1 L of purified water and stirring until complete dissolution. A surfactant solution was separately prepared by adding 12 g of polysorbate 80 to 750 mL of purified water. A separate concentrated chlorhexidine solution was also prepared by adding 10 g of chlorhexidine acetate to 50 mL of H₂O and stirring until complete dissolution.

The surfactant solution was added to the sodium alginate solution, and purified water was then added to the sodium alginate/surfactant solution to a final volume of 1950 mL. The concentrated chlorhexidine acetate solution was then added to the sodium alginate/surfactant solution at a controlled rate to produce a 2 L sodium alginate/surfactant/chlorhexidine solution. The temperature of all reaction vessels never exceed 35° C.

Microparticles were then formed from the sodium alginate/surfactant/chlorhexidine mixture by spray drying. More specifically, the solution was pumped into the spray dryer at a rate of between 1 and 10 mL/min. The inlet temperature was between 100° C. and 185° C. (average inlet temperature of 142.5° C.).

### Example 2—Chlorhexidine Elution

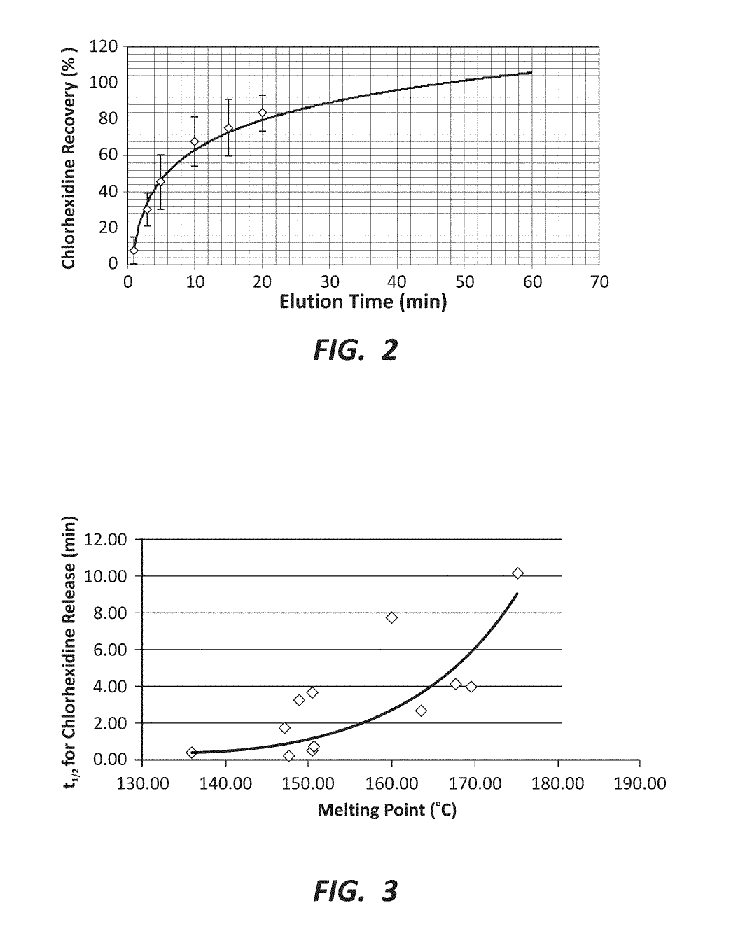

Chlorhexidine gluconate was incorporated into sodium alginate microspheres in a manner analogous to the process described above (0.075 g chlorhexidine gluconate per gram of microspheres). An elution profile was then generated (FIG. 2).

More particularly, the total amount of chlorhexidine in a sample of microparticles was determined by (1) treating a known mass of the microparticles with an aqueous HCl solution that caused complete release of chlorhexidine from the microparticles and (2) assessing the concentration of released chlorhexidine by HPLC. Next, an identical mass of microparticles was placed in a solution of 1× phosphate buffered saline (PBS), and the amount of eluted chlorhexidine was measured over time by HPLC. This process was repeated for multiple samples. The resulting elution profile is shown in FIG. 2.

### Example 3—Melting Point and Drug Elution Rate

The melting points for each of 11 batches of sodium alginate microparticles (i.e., the melting points for the sodium alginate in each batch) were determined by differential scanning calorimetry. The melting points were plotted against the t_{1/2} for chlorhexidine release, as determined by HPLC using the process described in connection with Example 2. The resulting plot is shown in FIG. 3.

As can be seen from FIG. 3, the drug elution rate generally correlates with the melting point of the sodium alginate microparticles. In other words, microparticles having a relatively high melting point tend to release chlorhexidine at a lower rate than microparticles with relatively low melting points.

### Example 4—SEM Images

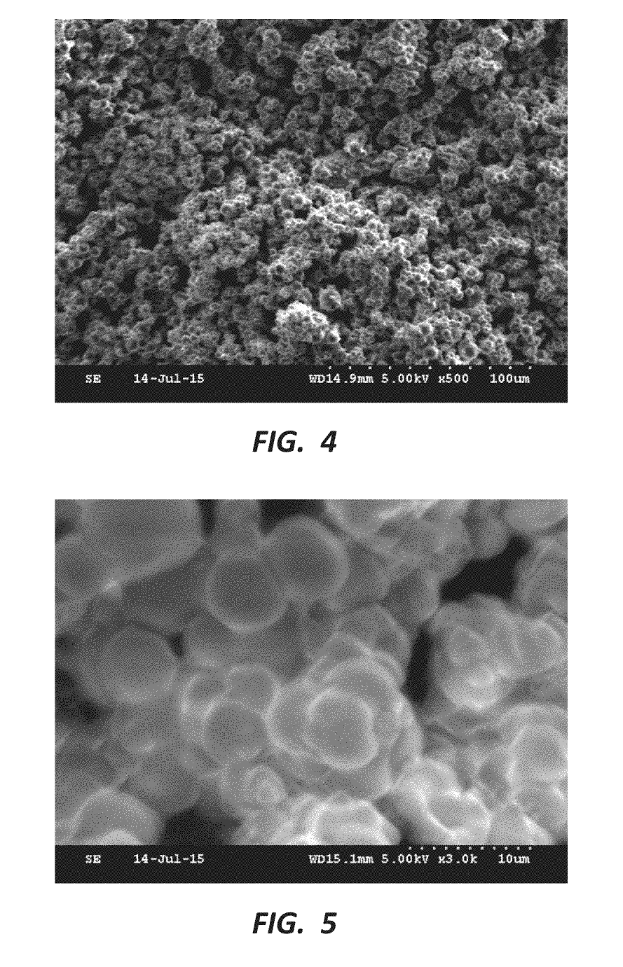

The microparticles manufactured as described in Example 1 were imaged using a scanning electron microscope (SEM). The resulting SEM images are shown in FIGS. 4 and 5. More particularly, FIG. 4 provides an image of the microparticles at 500× magnification, while FIG. 5 provides an image of the same microparticles at 3000× magnification. As can be seen from these images, the microparticles formed by the process of Example 1 were substantially spherical in shape.

### Example 5—Size Distribution of Microparticles

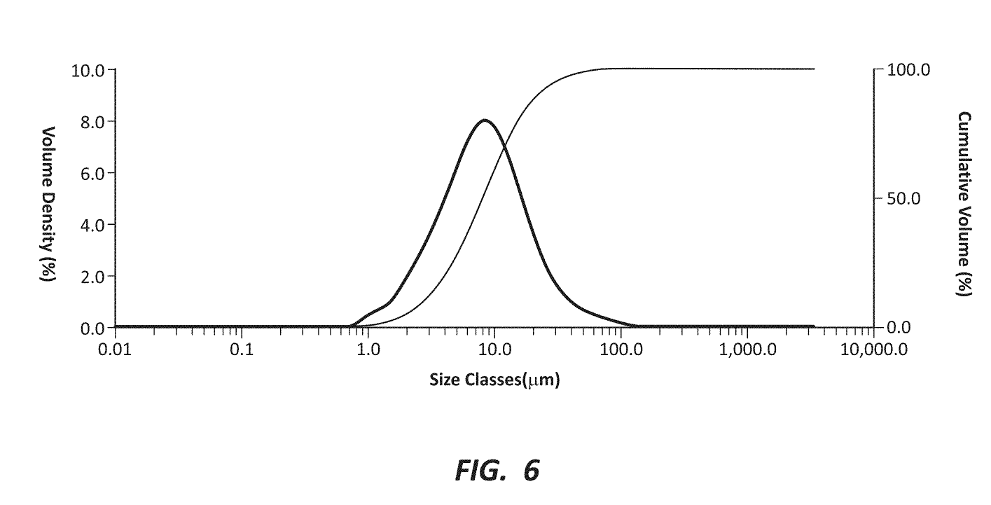

The size distribution of the microparticles of Example 1 was analyzed by laser diffraction. The resulting size distribution curves are shown in FIG. 6. As indicated on the y-axes of FIG. 6, the curves show the volume density and cumulative volume as a percentage of the total volume of the distribution. From this data, the D10, D50, and D90 diameters were determined. The D10 value (i.e., the total volume of the distribution that lies below the specified diameter) was 2.72 μm. The D50 value was 7.84 μm. And the D90 value was 21.2 μm. The D[4,3] value was determined to be 10.8 μm.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

It will be apparent to those having skill in the art that changes may be made to the details of the above-described embodiments without departing from the underlying principles of the present disclosure.

1. An antimicrobial composition comprising a plurality of microparticles, wherein each microparticle of the plurality of microparticles comprises:
a charged polysaccharide selected from the group consisting of chitosan, alginate, heparin, hyaluronic acid, and pectin; and
an antimicrobial agent.

2. The antimicrobial composition of claim 1, wherein the polysaccharide is sodium alginate.

3. The antimicrobial composition of claim 1, wherein the plurality of microparticles each have an alginate shell that encapsulates at least a portion of the antimicrobial agent.

4. The antimicrobial composition of claim 2, wherein the sodium alginate is cross-linked.

5. The antimicrobial composition of claim 1, wherein the antimicrobial agent comprises chlorhexidine.

6. The antimicrobial composition of claim 1, wherein the microparticles further comprise a surfactant.

7. The antimicrobial composition of claim 6, wherein the surfactant is a polysorbate.

8. The antimicrobial composition of claim 1, wherein the plurality of microparticles are substantially spherical in shape.

9. The antimicrobial composition of claim 1, wherein, on average, the microparticles of the plurality of microparticles are between 25% and 45% antimicrobial by weight.

10. The antimicrobial composition of claim 1, further comprising a foam, wherein the plurality of microparticles are disposed on an outer surface of the foam.

11. The antimicrobial composition of claim 1, wherein the antimicrobial composition is also a hemostatic composition.

12. A method for manufacturing a composition comprising a plurality of microparticles, the method comprising:
combining a negatively charged polysaccharide selected from the group consisting of alginate, heparin, hyaluronic acid, and pectin with a positively-charged antimicrobial agent, a surfactant, and a liquid to form a mixture; and
spray drying the mixture to form the plurality of microparticles.

13. The method of claim 12, wherein the negatively charged polysaccharide is sodium alginate.

14. The method of claim 12, wherein the liquid comprises water.

15. The method of claim 12, wherein spray drying the mixture removes more than 90% of the liquid in the mixture.

16. The method of claim 12, further comprising applying the plurality of microparticles to a substrate.

17. The method of claim 16, wherein the substrate is a dressing for a wound.

18. The method of claim 12, wherein the positively-charged antimicrobial comprises chlorhexidine.

19. The method of claim 12, wherein the mixture, immediately prior to spray drying, is between 0.2% and 2.5% positively charged antimicrobial agent (w/v).

20. The method of claim 12, further comprising combining a cross-linking agent with the negatively charged polysaccharide prior to spray drying the mixture.
